(19) United States
(12) Patent Application Publication
(10) Pub. No.: US 2015/0173625 A1
(43) Pub. Date: Jun. 25, 2015

(54) HIGH FREQUENCY ULTRASOUND TRANSDUCERS

(71) Applicant: FUJIFILM SonoSite, Inc., Bothell, WA (US)

(72) Inventors: Nicholas Christopher Chaggares, Whitby (CA); James Mehi, Thornhill (CA)

(21) Appl. No.: 14/577,185

(22) Filed: Dec. 19, 2014

Related U.S. Application Data
(60) Provisional application No. 61/919,163, filed on Dec. 20, 2013.

(51) Int. Cl. A61B 5/00; A61B 8/00

(52) U.S. Cl. CPC A61B 5/0095; A61B 8/4483

(57) ABSTRACT

High frequency ultrasound transducers configured for use with photoacoustics systems are disclosed herein. In one embodiment, an ultrasound transducer stack includes a transducer layer and an at least partially optically reflective lens layer. The lens can include a lens material doped with a plurality of optically reflective particles. In another embodiment, the transducer stack can further include a matching layer comprising a matrix material doped with a plurality of optically reflective particles. In a further embodiment, the transducer stack can include an optically reflective matching layer positioned proximate a front surface of an acoustic lens.

### CROSS REFERENCE TO RELATED APPLICATIONS

This application claims the benefit of U.S. Provisional Patent Application 61/919,163 filed on Dec. 20, 2013, which is incorporated by reference in its entirety.

### TECHNICAL FIELD

The disclosed technology generally relates to the fields of ultrasonic transducers and medical diagnostic imaging. More specifically, the disclosed technology relates to high frequency ultrasonic transducer stacks configured for use in photoacoustic imaging.

### PATENTS AND PATENT APPLICATIONS INCORPORATED BY REFERENCE

The following patents are also incorporated by reference herein in their entireties: U.S. Pat. No. 7,052,460, titled “SYSTEM FOR PRODUCING AN ULTRASOUND IMAGE USING LINE-BASED IMAGE RECONSTRUCTION,” and filed Dec. 15, 2003; U.S. Pat. No. 7,255,648, titled “HIGH FREQUENCY, HIGH FRAME-RATE ULTRASOUND IMAGING SYSTEM,” and filed Oct. 10, 2003; U.S. Pat. No. 7,230,368, titled “ARRAYED ULTRASOUND TRANSDUCER,” and filed Apr. 20, 2005; U.S. Pat. No. 7,808,156, titled “ULTRASONIC MATCHING LAYER AND TRANSDUCER,” and filed Mar. 2, 2006; U.S. Pat. No. 7,901,358, titled “HIGH FREQUENCY ARRAY ULTRASOUND SYSTEM,” and filed Nov. 2, 2006; and U.S. Pat. No. 8,316,518, titled “METHODS FOR MANUFACTURING ULTRASOUND TRANSDUCERS AND OTHER COMPONENTS,” and filed Sep. 18, 2009.

### BACKGROUND

Ultrasonic transducers convert electrical energy into acoustic energy and vice versa. When the electrical energy is in the form of a radio frequency (RF) signal, a properly designed transducer can produce ultrasonic signals having the same or similar frequency characteristics as the driving electrical RF signal. Diagnostic ultrasound has traditionally been used at center frequencies ranging from less than 1 MHz to about 10 MHz. One skilled in the art will appreciate that this frequency spectrum provides a capability to image biological tissue with a resolution ranging from, for example, several millimeters to greater than 300 microns, and at depths ranging, for example, from a millimeter to several centimeters.

High frequency ultrasonic (HFUS) transducers generally include ultrasonic transducers having center frequencies above 15 MHz and ranging to over 60 MHz. HFUS transducers can provide higher resolution while limiting the maximum depth of penetration, and as such, provide a means of imaging biological tissue from a depth of a fraction of a mm to over 3 cm with resolutions in the 20 um to 300 um range. There are many challenges associated with fabricating high frequency ultrasonic transducers that do not arise when working with traditional clinical ultrasonic transducers that operate at frequencies below about 10 MHz. One skilled in the art will appreciate that structures generally scale down according to the inverse of the frequency, so that a 50 MHz transducer will have structures about 10 times smaller than a 5 MHz transducer. In some cases, materials or techniques cannot be scaled down to the required size or shape, or in doing so they lose their function and new technologies must be developed or adapted to allow high frequency ultrasonic transducers to be realized. In other cases, entirely new requirements exist when dealing with the higher radio frequency electronic and acoustic signals associated with HFUS transducers.

Photoacoustic imaging is a modified form of ultrasound imaging that is based on the photoacoustic effect in which the absorption of electromagnetic energy (e.g., infrared light, visible light, ultraviolet light, radio-frequency waves, etc.) generates acoustic waves. In photoacoustic imaging, light pulses are transmitted into biological tissues, and a portion of the transmitted light energy is absorbed by tissues in a subject and converted into heat. The resulting heat can cause transient thermoelastic expansion, which can generate ultrasound waves. The generated ultrasonic waves are detected by ultrasonic transducers, which convert the received ultrasound waves into electrical signals used to form images.

One limitation of current photoacoustic systems is noise or artifacts in images formed using HFUS signals. Some of these artifacts are caused by transmitted laser light that is reflected by the skin of a subject back toward an HFUS transducer. The reflected light can be absorbed by one or more layers of the HFUS transducer and cause a secondary photoacoustic signal. The secondary photoacoustic signal shows up as an artifact in the photoacoustic image and, in many cases, can be stronger than the photoacoustic signals generated by light absorbed into the subject.

One approach to reduce secondary photoacoustic artifacts is to form several tomographic images by obtaining image data by rotating a transducer around a line normal to and located in the imaging plane. The resulting set of collected data taken at varied angles about the normal to the imaging plane can be combined through tomographic techniques reduce or eliminate non-coherent signals (e.g., noise, artifacts, etc.) between the angled data sets, thus forming images having little or no secondary artifact. As one skilled in the art will appreciate, however, a tomographic approach requires a subject to remain still for several seconds or more, and even then may take longer to acquire a single image. As a result, tomographic photoacoustic systems may not be practical in clinical or preclinical applications in which holding a subject still may not possible or desirable. In addition, observation of some anatomical functions, pharmacokinetics, or other dynamics may not be possible with the frame rate limitations inherent in multi-look approaches like tomography.

### BRIEF DESCRIPTION OF THE DRAWINGS

The invention may be more completely understood in consideration of the accompanying drawings, which are incorporated in and constitute a part of this specification, and together with the description, serve to illustrate the disclosed technology.

FIG. 1 is a schematic view of a photoacoustic imaging system configured in accordance with one or more embodiments of the disclosed technology.

FIG. 2 is a side schematic view of an ultrasound transducer configured in accordance with one or more embodiments of the disclosed technology.

FIG. 3 is a schematic view of an acoustic lens configured in accordance with an embodiment of the disclosed technology.

FIG. 4A is a schematic view of a transducer matching layer configured in accordance with an embodiment of the disclosed technology.

FIG. 4B is a schematic view of a transducer matching layer configured in accordance with another embodiment of the disclosed technology.

FIG. 5 is a schematic view of an ultrasound transducer configured in accordance with an embodiment of the disclosed technology.

FIG. 6 is a schematic view of an ultrasound transducer configured in accordance with another embodiment of the disclosed technology.

FIG. 7 is a schematic view of an ultrasound transducer configured in accordance with a further embodiment of the disclosed technology.

FIG. 8 is a schematic view of an ultrasound transducer configured in accordance with yet another embodiment of the disclosed technology.

### DETAILED DESCRIPTION

The technology disclosed herein generally relates to high frequency ultrasound transducers. In one aspect, a high frequency ultrasound transducer includes an acoustically penetrable, optically-reflective lens. The lens can be configured to have very low acoustic losses and sufficient acoustic lensing capability while exhibiting high reflectivity in an optical wavelength region of interest (e.g., 680-970 nanometers) while having low optical absorption in the same region. In one embodiment of this aspect, the optical reflectivity of the lens may be Lambertian (i.e., diffusively reflective). In some embodiments, a diffuse reflection may take place not only at the surface of the lens, but in a gradient extending into the surface of the lens, thus exhibiting a characteristic lying between truly opaque and having an opacity of less than 100%. A gradient based diffuse reflectivity can reduce or eliminate secondary photoacoustic artifacts as a result of reflected light. Moreover, in addition to opacity (i.e., a reduction of light transmission), it may be desirable to control a mechanism that prevents optical transmission such that light is reflected but not absorbed within the lens. Absorbed light will generally give rise to a photoacoustic effect that may lead to an artifact. Since it can be challenging to find a material that is 100% reflective with no significant acoustic absorption coefficient, other strategies may be employed to mitigate undesirable absorption artifacts in the lens. Accordingly, in one embodiment, a lens material (e.g., polymethylpentene) doped with reflective particles (e.g., titanium dioxide particles) can exhibit diffuse reflectivity with very low absorption while maintaining excellent acoustic transmission characteristics at high acoustic frequencies. In another embodiment, an optically-reflective coating (e.g., sputtered aluminum) can be applied to a surface (e.g., an underside surface) of an acoustic lens (e.g., a thermo set cross-linked polystyrene lens), thus preventing optical absorption of photons on the transducer stack behind the lens. In some embodiments, the lens includes between 90-95% of the matrix material and between 5-10% of the optically reflective material.

In another aspect of the present disclosure, an ultrasound transducer stack may include an optically reflective acoustic matching layer positioned behind (e.g., under) an acoustics lens. In one embodiment, the acoustic matching layer is configured to be at least partially opaque for the wavelengths used in the photoacoustic array. There are few materials suited for making HFUS acoustic matching layers that also have a high reflectivity in the optical wavelength range. An acoustic matching layer comprising, for example, a titanium dioxide powdered-loaded matrix may be suitable for use in HFUS arrays where a low to medium acoustic impedance (approximately 3 to 4 MR) is desired. In one embodiment, a matching layer may comprise an epoxy or glue doped with titanium dioxide at a ratio of 1:0.35 by weight (e.g., 1 g epoxy for 0.35 g of TiO₂). In other embodiments, for example, a hafnium dioxide and titanium dioxide powder mix may be suitable for use in HFUS arrays, where a medium acoustic impedance (e.g., between about 4 MR to about 6 MR) may be desired. Matching layers can be made opaque at relatively thin matching layer thicknesses (e.g., 25 microns or less). An opaque acoustic matching layer can reduce and/or mitigate secondary photoacoustic effects arising within the acoustic stack, even if the lens is optically transparent or only partially opaque.

In yet another aspect of the present disclosure, an external optically-reflective layer may be positioned in front of (e.g., on top of) an optically-transparent or highly-translucent acoustic lens. As discussed above, very few acoustic lens materials are optically reflective and acoustically transparent at frequencies associated with HFUS (e.g., 15 MHz or greater). If, however, the optically reflective layer at the front of the ultrasound stack is an acoustic matching layer, the acoustic losses may be disregarded, allowing for a larger selection of materials. Furthermore, those of ordinary skill in the art will appreciate that an acoustic lens material may be selected to provide as close an acoustic impedance match as possible to a medium to be imaged (e.g., tissue or water). A close impedance match may, for example, avoid unwanted multi-path reverberations between the acoustic lens and the acoustic objects in the field of the array.

In one embodiment of this aspect, an acoustic lens having a higher than typical acoustic impedance (e.g., between about 3 MR and about 5 MR) may be selected for use with the transducer stack to facilitate selection of the external optically-reflective layer. The external optically-reflective matching layer can be positioned in front of the lens and selected to have an acoustic impedance that is, for example, approximately the geometric mean of the lens and the tissue (e.g., less than 3 MR, between about 2 MR and about 3 MR, etc.). The external matching layer can be configured and/or selected to have excellent optical reflectance (e.g., greater than or equal to 50%, greater than or equal to 90%, etc.) and a thickness on the order of a fraction of an ultrasound wavelength (e.g., ¼ wave thick, ¾ wave thick, etc.) at frequencies associated with HFUS. Accordingly, in this embodiment, the external matching layer can be selected based on optical properties with less emphasis or consideration of acoustic losses. Correspondingly, the acoustic lens can be configured and/or selected based on acoustic lensing and attenuation characteristics, with less emphasis on optical characteristics of the acoustic lens.

Polybenzimidazole (hereinafter “PBI”) is one material that may be used to fabricate an acoustic lens having an optically reflective acoustic matching layer attached to the curved front of the lens. The external matching layer may comprise, for example a low acoustic impedance polymer (e.g., an optically transparent epoxy) doped with a light but highly optically reflective particle (e.g. TiO₂). This embodiment therefore requires no special consideration to the optical properties of acoustic layers behind the lens, as all optical energy is reflected from the front of the lens. In addition, an acoustic lens can be selected to have a relatively high speed of sound allowing the acoustic lens to have a relatively shallow curvature, thus mitigating an undesirable groove found on the face of conventional HFUS transducers. This property is generally useful for a matching layer placed in front of a higher speed of sound lens material (e.g., FBI) whether the external matching layer is optically reflective or not.

### Suitable Systems

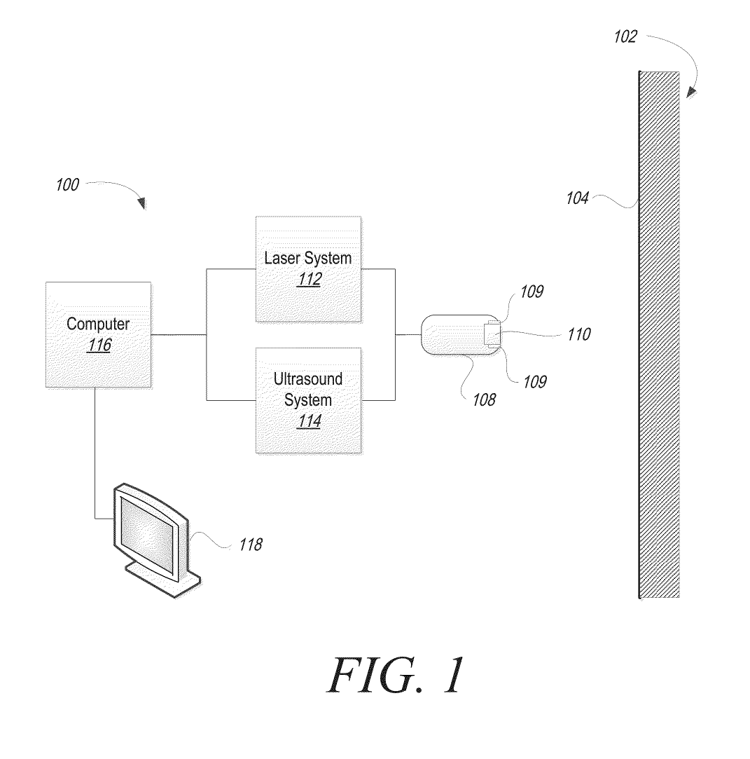

FIG. 1 is a schematic view of a photoacoustic imaging system 100 configured in accordance with embodiments of the disclosed technology. The system 100 includes a scanhead 108 configured to be placed at least proximate to a surface 104 (e.g., a skin line) of a target 102 (e.g., a patient, an animal, a small animal, a rat, a mouse, etc.). The scanhead 108 includes a plurality of optical fibers 109 and a transducer 110 positioned at a front portion of the scanhead 108. Portions of the optical fibers 109 can be positioned along one or more surfaces of the scanhead 108. In some embodiments, the optical fibers 109 can alternatively be integrated into the transducer 110. Holes drilled into portions of the transducer 110 (e.g., matching layers, acoustic lenses, etc.) can allow the optical fibers 109 and/or light emitted therefrom to pass through the transducer 110 unimpeded.

A laser system 112 is coupled to the optical fibers 109 and configured to produce electromagnetic (EM) energy (e.g., non-ionizing EM radiation, infrared light, visible light, ultraviolet light, etc.) An ultrasound system 114 is coupled (via, e.g., a wire, a wireless link, etc.) to the transducer 110 and is configured to generate high-frequency ultrasound (e.g., ultrasound energy having a center frequency of 15 MHz or greater). The ultrasound system 114 is also configured to receive high-frequency ultrasound echoes from the transducer 110. A computer 116 can receive the ultrasound signals (e.g., scan converted ultrasound signals) from the ultrasound system 114 and form one or more ultrasound images that can be presented to an operator via a display 118. One or more embodiments of the system 100 can include embodiments described in the applicants' co-pending U.S. patent application Ser. No. 13/695,275, which is incorporated by reference herein in its entirety.

In operation, the optical fibers 109 can transmit and direct laser light pulses (e.g., light pulses having wavelengths between approximately 680 nm and 970 nm) from the laser system 112 toward the one or more tissue structures (e.g., a heart, one or more blood vessels, a kidney, a uterus, a prostate, etc.) in and/or at the target 102. As those of ordinary skill in the art will appreciate, at least a portion of the laser light can be absorbed by the one or more tissue structures and converted into heat. The converted heat can cause a thermoelastic expansion in the tissue and a corresponding emission of acoustic energy (e.g., ultrasound energy). The transducer 110 receives the resulting ultrasound echoes from the target 102 and converts them into ultrasound signals. The computer 116 can include a memory and/or one or more processors configured to process the ultrasound signals and form one or more ultrasound images.

### Suitable Ultrasound Transducers

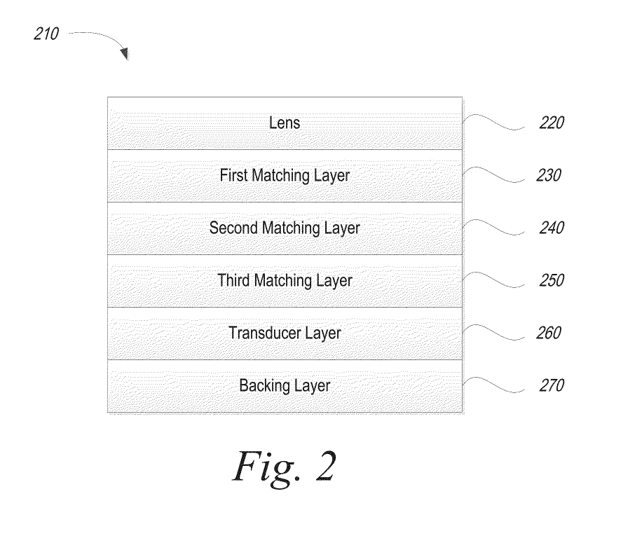

FIG. 2 is a schematic view of an ultrasound transducer 210 configured in accordance with embodiments of the disclosed technology. In the illustrated embodiment, the transducer 210 includes a plurality of layers—including a lens layer 220, a third matching layer 230, second matching layer 240, a first matching layer 250, a transducer layer 260, and a backing layer 270—each having a first surface (e.g., an lower surface) and a second surface (e.g., a upper surface). In some embodiments, however, a single matching layer (e.g., the first matching layer 250) may be implemented in the transducer 210 (e.g., between the lens layer 220 and the transducer 260). In other embodiments, for example, more than three matching layers may be implemented in the transducer 210. In further embodiments, the transducer 210 may not include any matching layers and may instead include, for example a lens layer (e.g., the lens layer 220) bonded directly to the transducer layer 260. In addition, components typically associated with ultrasound transducers (e.g., electrical interconnects, wires, circuits, printed circuit boards, active cooling devices, thermally conductive structures, kerfs separating individual transducer elements, etc.) are hidden in FIG. 2 for the sake of clarity.

The transducer layer 260 can comprise any suitable transducer material capable of transmitting and/or receiving high frequency ultrasound [e.g., piezoelectric transducers (e.g., lithium niobate transducers), capacitive micromachined ultrasound transducers (CMUTs), piezoelectric micromachined ultrasound transducers (PMUTs), etc.]. The transducer layer 260 can comprise one transducer (e.g., a single element transducer) or a plurality of transducers (e.g., a one-dimensional array of transducer elements and/or a multi-dimensional array of transducer elements). In some embodiments, the transducer layer 260 can comprise one or more additional transducer layers (not shown). The transducer layer 260 is configured to transmit and receive ultrasound energy at frequencies greater than 15 MHz. In one embodiment, the transducer layer 260 may comprise a transducer described in, for example, U.S. Pat. No. 7,230,368 and U.S. patent application Ser. No. 11/109,986 which are incorporated by reference herein in their entireties.

The backing layer 270 underlies the transducer layer 260, and can be configured to absorb rear-propagated acoustic energy and/or thermal energy produced by the transducer 210. Suitable backing layers are described in U.S. Pat. No. 7,750,536 and U.S. patent application Ser. No. 11/366,953 which are incorporated by reference herein in their entireties. In some embodiments (not shown), one or more layers (e.g., a dematching layer) can be disposed between the transducer layer 260 and the backing layer 270.

In the embodiment illustrated in FIG. 2, the lens layer 220 includes a lower surface overlaying an upper surface of the third matching layer 230. The lens layer 220 can be configured, for example as a thin film (e.g., having a thickness less than 50 microns) and can comprise a material that is acoustically transparent at high frequencies (e.g., polymethylpentene, thermo-set cross-linked polystyrene, a plastic, a polymer and/or a combination thereof). The lens layer 220 can also be configured to provide an acoustical impedance closely matched to water or another medium of interest. The lens layer 220 can have an acoustical impedance, for example, ranging from about 1 Megarayl (MR) to about 4 MR, ranging from about 1.5 MR to about 3 MR, or approximately 1.8 MR. In the illustrated embodiment, the lens layer 220 is shown having a flat upper surface (e.g., an outer and/or exterior surface). In other embodiments, however, the lens layer 220 may comprise a curved upper surface.

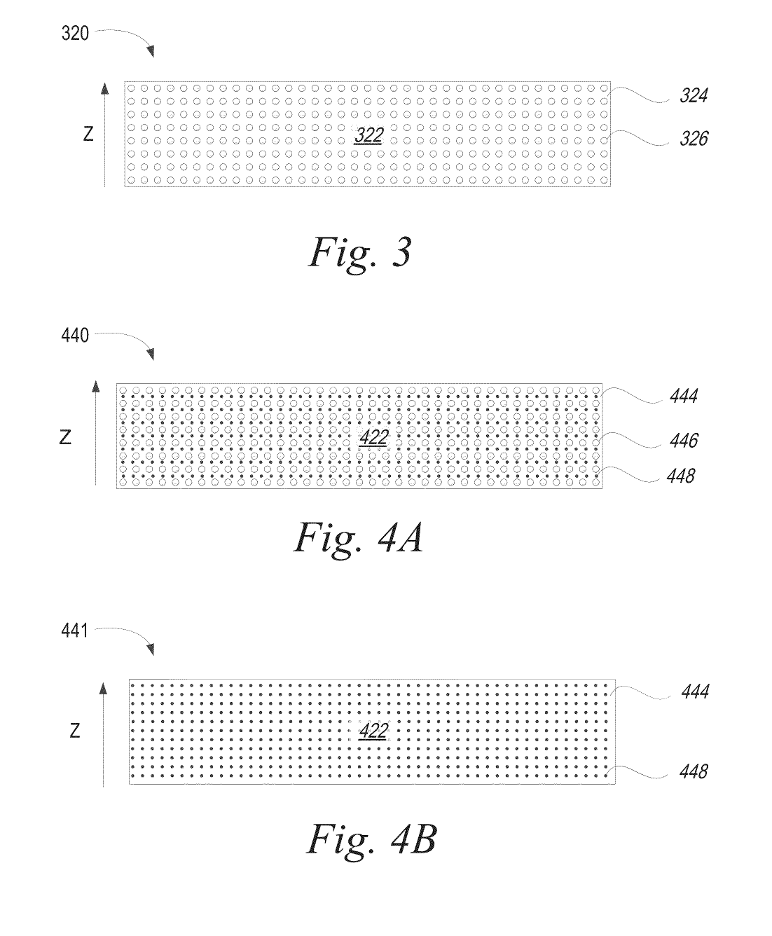

In one aspect of the disclosed technology (described in more detail below with reference to FIG. 3), the lens layer 220 can comprise a composite material that includes a matrix material (e.g., polymethylpentene) doped with particles of one or more materials. In some embodiments, for example, the lens layer 220 can be doped with particles of an optically-reflective material (e.g., TiO₂ and/or another suitable material capable of reflecting optical energy having wavelengths between about 680 nm and about 970 nm). Doping the lens layer 220 with optically-reflective particles can provide at least an advantage of reflecting optically energy away from the transducer 210. As those of ordinary skill in the art will appreciate, if an optically-absorptive matching layers underlie the lens layer 220, optical energy may be absorbed by the matching layer, thereby causing a secondary photoacoustic effect within the matching layer itself. The secondary photoacoustic effect and cause an emission of ultrasound energy that can cause significant noise or otherwise interfere with ultrasound echoes received at the transducer layer 260 from the subject.

As shown in FIG. 2, the first matching layer 230, the second matching layer 240, and the third matching layer 250 (collectively referred to hereinafter as “the matching layers 230-250”) are disposed between the lens layer 220 and the transducer layer 260. The matching layers 230-250 can be made from a variety of materials that are acoustically transparent at high frequencies (e.g., 15 MHz or greater) such as, for example, an epoxy, a polymer, etc. In one embodiment, for example, the first matching layer 230 can comprise a material (e.g., cyanoacrylate) capable of bonding the lens layer 220 (e.g., a lens layer made of polymethylpentene) to the second matching layer 240 (e.g., a low-viscosity epoxy matching layer). In some embodiments, the matching layers 230-250 can include one or more matching layers described in, for example, U.S. Pat. No. 7,750,536 and U.S. patent application Ser. No. 11/366,953, which are incorporated by reference herein in their entireties.

The matching layers 230-250 can be configured to provide and/or improve an impedance match between the lens layer 220 and the transducer layer 260. As those of ordinary skill in the art will appreciate, the transducer layer 260 may have, for example a relatively high acoustic impedance (e.g., greater than 10 MR) while the lens layer 220 may have an acoustical impedance (e.g., 1.5-2.5 MR) relatively similar to a subject being imaged (e.g., the target 102 of FIG. 1). Accordingly, the matching layers 230-250 can be configured to provide an impedance transition or gradient between the transducer layer 260 to the lens layer 220. The individual matching layers 230-250 can have, for example, gradually decreasing acoustic impedances. For example, the third matching layer 250 can have an acoustic impedance of between about 7.0 MR and about 14.0 MR. The second matching layer 240 can have an acoustic impedance of between about 3.0 MR and about 7.0 MR. The third matching layer 230 can have an acoustic impedance of between about 2.5 MR and about 2.8 MR. Moreover, in some embodiments, each of the matching layers 230-250 can be a ¼ wavelength matching layer. In other embodiments, however, individual matching layers 230-250 can have a thickness corresponding to any fractional ultrasound wavelengths (e.g., ½, ¼, ⅛, 1/16 etc.). In further embodiments, the matching layers 230-250 can have any suitable thickness.

In one aspect of the present technology, one or more of the matching layers 230-250 can comprise a composite material that includes a matrix material (e.g., a polymer) and a plurality of first and second particles. In some embodiments, for example, the first particles may comprise a first material having a first density, and the second particles may comprise a second material having a second density less than the first density. The composite material may be formed by adding the first particles in a first amount to the matrix material until a desired density and/or acoustical impedance of the composite material is achieved. The second particles may be selected based on, for example, such that the second density of the second particles is substantially similar and/or identical to the desired density of the composite material. The second particles may be therefore be added to the composite material in a second amount until a desired consistency, homogeneity, viscosity, and/or thixotropic index of the composite material achieved. Because the second density is substantially similar to the desired density of the composite material, the second particles can be added without significantly altering the density and, thus, the acoustical impedance of the composite material. In another aspect of the present technology, as described in, for example, U.S. Pat. No. 7,750,536, the first particles can include micron-sized particles and the second particles can include nano-sized particles. In yet another aspect of the present technology, as described in detail below with reference to FIG. 4, the first particles and second particles may comprise substantially optically reflective materials.

FIG. 3 is a schematic view of an acoustic lens layer 320 configured in accordance with an embodiment of the disclosed technology. In the illustrated embodiment, the lens layer 320 (e.g., the lens layer 220 of FIG. 2) comprises a composite material 322 that includes a matrix material 324 doped with a plurality of first particles 326. The matrix material 324 may comprise, for example, a durable lens material that is substantially acoustically transparent at high frequencies (e.g., 15 MHz or greater) while also having a suitable acoustic impedance (e.g., between about 1.0 MR and 4.0 MR). In some embodiments, for example, the matrix material 324 may comprise polymethylpentene and/or thermo-set cross-linked polystyrene.

The first particles 326 can comprise an optically reflective material (e.g., TiO₂, a white pigment, etc.) that, within a range of concentration (e.g., between about 1% and about 20%), is also substantially acoustically transparent at high frequencies. The first particles 326 can have a diameter significantly small to allow, for example, multiple grain heights along the z-direction of the lens layer 326. In some embodiments, for example, the diameter may be less than 5 microns or between about 2 and 3 microns. In other embodiments, however, the first particles 326 may have any suitable diameter. Further, the first particles 326 may comprise a material having a density substantially similar to the density of the matrix material 324 such that the composite material 322 has a density (and thus, an acoustical impedance) substantially similar to the matrix material 324.

The first particles 326 may be doped or otherwise loaded into the matrix material 324 in a first amount (e.g., a volumetric ratio of 5%, 10%, 20%, 30%, 40%, etc.) to achieve a desired reflectance (e.g., greater than 90% at EM wavelengths between about 680 nm and 970 nm within the thickness of the lens) of the composite material 322, while remaining substantially acoustically transparent at high frequencies. Moreover, in the illustrated embodiment of FIG. 3, the first particles 326 are shown as a substantially homogeneous distribution of particles within the matrix material 324. In some embodiments, however, the first particles 326 may be arranged to provide a gradient of optical reflectivity such that the reflectivity increases or decreases within the lens layer 320 along the z-direction. In other embodiments, for example, the first particles 326 can be arranged within the matrix material 324 in any suitable fashion.

As those of ordinary skill in the art will appreciate, a transducer configured for use with low frequency ultrasound (e.g., 10 MHz or less) can include a relatively thick acoustic lens (e.g., 250 microns or greater) having sufficient opacity to resist the secondary photoacoustic effects described above. On the contrary, a transducer configured for use with high frequency ultrasound (e.g., 15 MHz or greater) may require an acoustic lens having a relatively low thickness (e.g., 100 microns or less) and attenuation. Acoustic lenses suitable for use with high-frequency ultrasound are typically formed as optically-transparent films that allow virtually all incoming light to pass therethrough. As noted above, light entering the transducer (e.g., the transducer 210 of FIG. 2) can cause significant noise and artifacts in an ultrasound image as a result of secondary photoacoustics effects that may occur when laser light (e.g., laser light from the laser system 112 of FIG. 1) is reflected toward an acoustic lens (e.g., by the surface 104 of FIG. 1). A substantially acoustically-transparent and optically-reflective lens layer (e.g., the lens layer 320) can provide at least an advantage of preventing, reducing and/or mitigating these secondary photoacoustic effects in high frequency ultrasound transducers.

FIG. 4A is a schematic view of a matching layer 440 configured in accordance with an embodiment of the disclosed technology. The matching layer 440 can have a thickness corresponding to a fraction (e.g., ½, ¼, ⅛, 1/16 etc.) of suitable ultrasound wavelengths (e.g., wavelengths corresponding to ultrasound frequencies of 15 MHz or greater). In the illustrated embodiment, the matching layer 440 (e.g., the second matching layer 240 of FIG. 2) comprises a composite material 422 that includes a matrix material 444, first particles 446 and a second particles 448. The matrix material 444 can comprise, for example, a polymer (e.g., an epoxy, EPO-TEK® 301 or 302, Cotronics Duralco® 4461, etc.) or a thermoplastic such as, for example, polymethylmethacrylate (PMMA), acrylic, PLEXIGLAS®, LUCITE® and/or polycarbonate (PC). Additional suitable matrix materials may be found in, for example, U.S. Pat. No. 7,750,536.

The first particles 446 can comprise, for example, a first optically-reflective powder (e.g., hafnium oxide) selected to have a high density much higher than the density of the composite material 442 which has an acoustic impedance between about 4.0 MR and about 7 MR). The second particles 448 can comprise a second optically-reflective powder (e.g., TiO₂, a white powder, a white pigment, and/or any suitable optically reflective material) having a density substantially similar to the desired density of the composite material. The second particles 448 can thus be added relatively freely without significantly changing the density of the composite, allowing a designer to vary the viscosity and reflectance somewhat independently from the acoustic impedance (which is a product of the density and speed of sound). In some embodiments, the first particles 446 and the second particles 448 may comprise the same material. In one embodiment, for example, the first particles 446 can have a first diameter ranging from about 2.0 microns to about 6 microns, and the second particles 448 can have a second diameter ranging from about 0.5 microns to about 0.9 microns. In some embodiments, however, either the first particles 446 or the second particles 448 may have diameters substantially less than 1.0 micron (e.g., nano-sized particles). Moreover, in the embodiment illustrated in FIG. 4, the first particles 446 and the second particles 448 are shown. In other embodiments, however, the matching layer 440 may include only the first particles 446. In further embodiments, the matching layer 440 may include particles of three or more materials. In still further embodiments, the matching layer 440 may include only the matrix material 444 without particles loaded therein.

The first particles 446 can be loaded into the matrix material 442 in a first amount (e.g., 60% by weight) and the second particles 448 can be loaded into the matrix material 442 in a second amount (e.g., 10% by weight) to achieve a desired reflectance (e.g., greater than 90% at EM wavelengths between about 680 nm and 970 nm) and/or desired acoustic impedance of the composite material 442. Both sets of particles may be implemented, for example, because the first particles 446 (e.g., Hafnium Oxide) may have a desirable density that can increase or decrease an acoustical impedance of the composited material 442, and may be at least partially optical reflective when loaded into the matrix material 444. However, the resulting composite material with only the matrix material 444 and the first particles 446 may not be sufficient to achieve a desired reflectance (e.g., greater than 90%). Adding the second particles 448 to the matrix material 444 with the first particles 446 can result in the composite material 442 having the desired reflectance without significantly affecting the acoustical performance of the matching layer 440. A composite layer 442 having a sufficient high reflectance can provide at least an advantage of preventing, reducing and/or mitigating secondary photoacoustic effects in high frequency ultrasound transducers, as discussed above in reference to the lens layer 320 of FIG. 3.

In some embodiments, as shown in FIG. 4B, a matching layer 441 may include the second particles 448 (e.g., TiO₂ particles and/or any suitable highly reflective particles) without the first particles 446 if, for example, the density of the composite material 442 does not require a substantial adjustment. Thus, the reflectance and opacity of the matching layer 441 can be determined by, for example, a wetting limit of the matrix material and/or the viscosity limits of the uncured composite material 442.

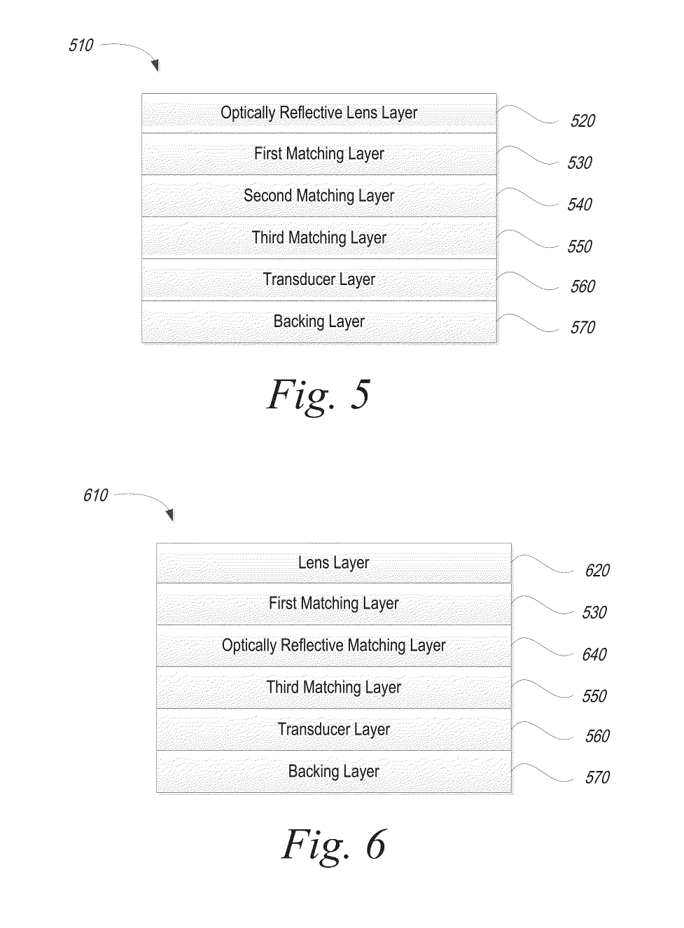

FIG. 5 is a schematic view of an ultrasound transducer 510 configured in accordance with an embodiment of the disclosed technology. In the illustrated embodiment, the transducer 510 (e.g., the transducer 210 of FIG. 2) includes a plurality of layers each having a first surface (e.g., an upper surface) and a second surface (e.g., a lower surface). The transducer 510 includes an optically reflective acoustic lens 520 (e.g., the lens 320 of FIG. 3), a first matching layer 530 (e.g., a ¼-wavelength cyanoacrylate matching layer), an optically reflective matching layer 540 (e.g., the matching layer 440 of FIG. 4), a third matching layer 550 (e.g., an optically absorptive matching layer), a transducer layer 560 (e.g., the transducer layer 260 of FIG. 2), and a backing layer 570 (e.g., the backing layer 570 of FIG. 2). Moreover, the optically reflective lens 520 may be loaded with an optically reflective particles (e.g., the first particles 326 of FIG. 3) to provide an optimal compromise between acoustic transparency and optical reflectivity, such that some optical energy is allowed to pass through the lens to be subsequently reflected by a highly optically reflective acoustic matching 540 layer while minimizing acoustic loss and associated heating of the lens.

FIG. 6 is a schematic view of an ultrasound transducer 610 configured in accordance with another embodiment of the disclosed technology. In the illustrated embodiment, the transducer 610 (e.g., the transducer 210 of FIG. 2) includes a plurality of layers each having a first surface (e.g., an upper surface) and a second surface (e.g., a lower surface). The transducer 610 includes an acoustic lens 620 (e.g., a polymethylpentene lens), the first matching layer 530, an optically reflective matching layer 640 (e.g., the matching layer 440 of FIG. 4), the third matching layer 550, the transducer layer 560, and the backing layer 570.

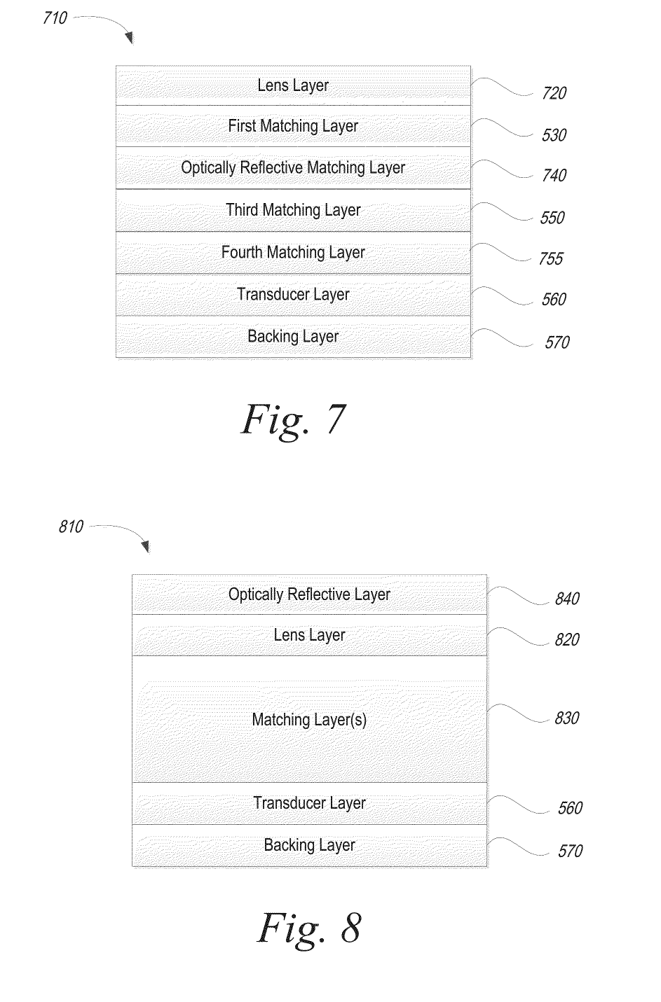

FIG. 7 is a schematic view of an ultrasound transducer 710 configured in accordance with a further embodiment of the disclosed technology. In the illustrated embodiment, the transducer 710 includes a plurality of layers each having a first surface (e.g., an upper surface) and a second surface (e.g., a lower surface). The transducer 710 includes an acoustic lens 720 (e.g., a thermo set cross-linked polystyrene lens), the first matching layer 530, an optically reflective matching layer 740 (e.g., the matching layer 440 of FIG. 4), the third matching layer 550, a fourth matching layer 755, the transducer layer 560, and the backing layer 570.

FIG. 8 is a schematic view of an ultrasound transducer 810 configured in accordance with yet another embodiment of the disclosed technology. In the illustrated embodiment, the transducer 810 includes a plurality of layers each having a first surface (e.g., an upper surface) and a second surface (e.g., a lower surface). The transducer 810 includes an optically reflective layer 840 (e.g., the optically reflective matching layer 740 of FIG. 7) positioned proximate (e.g., in front or on top of) an acoustic lens 820 (e.g., a lens comprising PBI, a metal, a thermoplastic, a polymer, polymethylpentene, a thermo set cross-linked polystyrene, etc.) that may be substantially optically transparent or opaque, but substantially acoustically transparent to HFUS. One or more matching layers 830 are positioned between the acoustic lens 820 and the transducer layer 560. In some embodiments, the matching layers 830 may include a second matching layer (e.g., the first matching layer 530 of FIG. 5). In certain embodiments, the matching layers 830 may include additional matching layers (e.g., the third matching layer 550 of FIG. 5 and/or the fourth matching layer 755 of FIG. 7).

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. An ultrasound transducer, comprising:
an acoustically penetrable lens layer having a lower surface, wherein the lens layer is at least partially optically reflective; and
a transducer layer underlying the lower surface of the lens layer, wherein the transducer layer is configured to receive ultrasound energy from a subject.

2. The ultrasound transducer of claim 1 wherein the lens layer comprises a composite material that includes a matrix material doped with particles of an optically reflective material.

3. The ultrasound transducer of claim 2 wherein the matrix material comprises polymethylpentene, and wherein the optically reflective material comprises titanium dioxide.

4. The ultrasound transducer of claim 2 wherein the composite material includes between 90-95% of the matrix material and between 5-10% of the optically reflective material.

5. The ultrasound transducer of claim 2 wherein the particles of the optically reflective material have a diameter less than 5 microns.

6. The ultrasound transducer of claim 2 wherein the particles of the optically reflective material have a diameter between about 2 to 3 microns.

7. The ultrasound transducer of claim 1 wherein the transducer layer is configured to receive ultrasound energy at frequencies of about 15 MHz or greater.

8. The ultrasound transducer of claim 1 wherein the lens layer is configured to at least partially reflect electromagnetic energy having wavelengths between 680 nanometers and 970 nanometers.

9. The ultrasound transducer of claim 8 wherein the lens layer has a reflectance of about 90% or greater.

10. The ultrasound transducer of claim 1, further comprising a first matching layer and a second matching layer each having an upper surface opposing a lower surface, wherein the upper surface of the first matching layer underlies the lower surface of the lens, wherein the upper surface of the second matching layer underlies the lower surface of the first matching layer, and wherein the upper surface of the transducer layer underlies the lower surface of the second matching layer.

11. The ultrasound transducer of claim 10 wherein the first matching layer comprises cyanoacrylate.

12. The ultrasound transducer of claim 10 wherein the second matching layer comprises a second composite material that includes a second matrix material, a first powder and a second powder.

13. The ultrasound transducer of claim 12 wherein the first powder comprises hafnium dioxide and the second powder comprises titanium dioxide.

14. The ultrasound transducer of claim 12 wherein the first powder and the second powder are at least partially optical reflective.

15. The ultrasound transducer of claim 12 wherein a first amount of the first powder is combined with the second matrix material such that the second composite material has a desired acoustic impedance.

16. The ultrasound transducer of claim 15 wherein a second amount of the second powder is combined with the second matrix material and the first powder to maintain the consistency, viscosity and thixotropic index of the resultant second composite material.

17. The ultrasound transducer of claim 12 wherein the second powder has an acoustic impedance generally similar to a desired acoustic impedance of the composite material.

18. The ultrasound transducer of claim 12 wherein the first powder comprises a plurality of first particles, wherein the second powder comprises a plurality of second particles, and wherein individual first particles are heavier than individual second particles.

19. The ultrasound transducer of claim 12 wherein the first powder comprises a plurality of first particles, wherein the second powder comprises a plurality of second particles, and wherein the individual first particles have a first diameter greater than a second diameter of the individual second particles.

20. The ultrasound transducer of claim 10 wherein the first and the second matching layers are ¼-wavelength matching layers.

21. A photoacoustics system, comprising:
a laser system configured to generate laser light pulses;
one or more optical fibers configured to direct the laser light pulses toward a target; and
an ultrasound transducer that includes—
a first matching layer comprising a composite material that includes a matrix material and a powder, wherein the powder is at least partially optical reflective, and wherein the composite material is substantially acoustically transparent at frequencies greater than 15 MHz.
a transducer layer underlying the first matching layer, wherein the transducer layer is configured to receive ultrasound energy at frequencies of 15 MHz or greater from a subject.

22. The photoacoustics system of claim 21, further comprising a lens layer overlying the first matching layer and comprising a matrix material doped with particles of an optically reflective material.

23. The photoacoustics system of claim 22 wherein the matrix material comprises polymethylpentene, and wherein the optically reflective material comprises titanium dioxide.

24. The photoacoustics system of claim 21 wherein the first matching layer is configured to at least partially reflect electromagnetic energy having wavelengths between 680 nanometers and 970 nanometers.

25. The ultrasound transducer of claim 24 wherein the first matching layer has a reflectance of 90% or greater.

26. The photoacoustics system of claim 22, further comprising a second matching layer disposed between the first matching layer and the lens layer, wherein the second matching layer comprises cyanoacrylate.

27. The photoacoustics system of claim 26 wherein the first and second matching layers are ¼-wavelength matching layers

28. The photoacoustics system of claim 21 wherein the powder comprises a first powder, and further comprising a second powder.

29. The photoacoustics system of claim 28 wherein the first powder comprises titanium dioxide and the second powder comprises hafnium dioxide.

30. The photoacoustics system of claim 21, further comprising an acoustic lens layer positioned between the first matching layer and the transducer layer.

31. An ultrasound transducer, comprising:
an acoustically penetrable lens layer having an upper surface; and
a matching layer positioned proximate the upper surface of the lens layer, wherein the matching layer is substantially optically reflective.

32. The ultrasound transducer of claim 31 wherein the lens layer is substantially optically transparent.

33. The ultrasound transducer of claim 31 wherein the lens layer comprises polybenzimidazole.

34. The ultrasound transducer of claim 31 wherein the matching layer has an optical reflectance of about 90% or greater.

35. The ultrasound transducer of claim 31 wherein the matching layer comprises a composite material that includes a matrix material and a powder, and wherein the composite material is substantially acoustically transparent at frequencies greater than 15 MHz.

36. The ultrasound transducer of claim 35 wherein the matrix material comprises an epoxy and wherein the powder comprises titanium dioxide.

37. The ultrasound transducer of claim 31 wherein the lens layer has an acoustical impedance greater than 3 MR, and wherein the matching layer has an acoustical impedance less than the lens layer.

38. The ultrasound transducer of claim 31, further comprising a transducer layer underlying the lens layer, wherein the transducer layer is configured to receive ultrasound energy from a subject at frequencies of about 15 MHz and greater.
